(19) United States
(12) Patent Application Publication
(10) Pub. No.: US 2004/0039368 A1
(43) Pub. Date: Feb. 26, 2004

(54) Devices, systems and methods for injecting multiple fluids into a patient

(76) Inventors: David M. Reilly, Glenshaw, PA (US); David M. Griffiths, Pittbugh, PA (US)

Correspondence Address: GREGORY L BRADLEY, MEDRAD INC, ONE MEDRAD DRIVE, INDIANOLA, PA, 15051

(21) Appl. No.: 10/463,739

(22) Filed: Jun. 17, 2003

Related U.S. Application Data
(60) Provisional application No. 60/391,455, filed on Jun. 25, 2002.

(51) Int. Cl. A61M 31/00

(52) U.S. Cl. 604/500; 604/151; 604/191; 604/82

(57) ABSTRACT

An adapter for connecting a syringe system having a plurality of syringe barrels to an injector having a drive member. The adapter includes an injector attachment, an intermediate drive member including a plurality of plunger drives, and a syringe system attachment. Preferably, each of the plurality of plunger drives is in general alignment with one of the plurality of barrels when the syringe system is attached to the adapter via the syringe system attachment.

### CROSS REFERENCE TO RELATED APPLICATION

This application claims the benefit of U.S. Provisional Application Serial No. 60/391,455, filed on Jun. 25, 2002, the contents of which are incorporated herein by reference.

### BACKGROUND OF THE INVENTION

The present invention relates generally to devices, systems and methods for injecting multiple fluids into a patient and, especially, to devices, systems and methods for generally simultaneously injecting multiple fluids into a patient from multiple syringe chambers or barrels.

In certain medical procedures, it is desirable to separately, but simultaneously, inject more than one injection fluid into a patient. For example, it may be desirable to simultaneously inject one or more fluids (which can be the same or different) into separate injection sites on a patient.

Moreover, it may in certain situations be desirable to generally simultaneously inject different fluids into one or more injection sites on a patient wherein the fluids cannot be mixed prior to injection. For example, U.S. Pat. No. 6,290,689 discloses a catheter and methods of use thereof for treatment of calcified vascular occlusions in which a vascular site is flushed simultaneously with an acidic dissolution fluid and a buffer solution.

In addition, there may be certain situations where two fluids are administered where the fluids must be combined to form an activated substance for treatment of a patient.

Use of powered injector systems to effect the simultaneous injection of multiple fluids as described above can afford a number of advantages over current manual injection procedures, including, but not limited to, improved control over injection variables or parameters, improved automation (with attendant reduction in operator tasks/time), and reduction in the risk of operator error. In that regard, a number of powered injector systems for use in medical procedures such as angiography, computed tomography, ultrasound and magnetic resonance imaging (MRI) are commercially available. Typically, such injectors include drive members such as pistons that operatively connect to a syringe plunger of a syringe attached to the injector. A front-loading syringe and injector system is disclosed, for example, in U.S. Pat. No. 5,383,858, the disclosure of which is incorporated herein by reference.

In currently available injectors systems, simultaneous injection of multiple, unmixed fluids would generally require use of multiple syringes on multiple injectors or injectors having multiple drive members. However, resources such as powered injectors are limited and costly, and multiple injectors may not be available in a facility for a single injection procedure. Moreover, use of multiple injectors or injectors equipped with multiple drive members can require significant operator setup time.

It is, therefore, desirable to develop devices, systems and methods whereby multiple fluids can be generally simultaneously injected into a patient via powered injection using a single injector. In addition, it is also desirable to develop devices, systems and methods for simultaneous injection and aspiration of fluids.

### SUMMARY OF THE INVENTION

In one aspect, the present invention provides an adapter for connecting a syringe system having a plurality of syringe barrels to an injector having a drive member. The adapter includes an injector attachment, an intermediate drive member including a plurality of plunger drives, and a syringe system attachment. In one embodiment, each of the plurality of plunger drives is in general alignment with a respective one of the plurality of barrels when the syringe system is attached to the adapter via the syringe system attachment.

The syringe system can, for example, include a plurality of separate syringes. The plurality of syringe barrels of the syringe system can also be in generally parallel attachment.

In a number of embodiments, the injector attachment comprises at least one flange positioned on a rear portion of the adapter. The syringe system attachment can, for example, include at least one retaining member that is movable to move into and out of abutting engagement with the syringe system. The syringe system attachment can also include a fixed retaining member to engage the syringe.

The intermediate drive member can include a common member to which the plurality of plunger drives is in operative connection. The common member can include a drive attachment for removably attaching the intermediate drive to the drive member of the injector.

In another aspect, the present invention provides a system for generally simultaneous injection of a plurality of injection fluids. The system includes a syringe system having a plurality of barrels, wherein each syringe barrel has a plunger slidably disposed therein. The system also includes an injector including a drive member, and an attachment interface. The system further includes an adapter generally as described above. The adapter includes an injector attachment cooperating with the attachment interface of the injector to attach the adapter to the injector; an intermediate drive member having a plurality of plunger drives, and a syringe system attachment. The intermediate drive member translates at least forward motion of the drive member of the injector to the syringe plungers. In one embodiment, each of the plurality of plunger drives of the intermediate drive member is preferably in general alignment with a respective one of the plurality of syringe barrels when the syringe system is attached to the adapter via the syringe system attachment.

In another aspect, the present invention provides a method of generally simultaneous injection of a plurality of injection fluids (which can be the same or different) using an injector including a drive member. The method comprises: placing an intermediate drive member in operative connection with the drive member of the injector, the intermediate drive member including a plurality of plunger drives; and advancing the drive member of the injector to generally simultaneously advance each of the plurality of plunger drives to pressurize a fluid in each of a plurality of syringe barrels with which the plunger drives are in operative connection.

The adapters or connection of the present invention allow the use of a single, currently available injector to effect the generally simultaneous injection of multiple fluids (which can be the same or different) into a patient. Such simultaneous injection can, for example, be used during high flow contrast enhanced CT diagnostic imaging exams, or during injection of contrast agent and inotropic agents associated with cardiac stress exams. As described in U.S. patent application Ser. No. 10/159,592, filed on May 30, 2002, and entitled FRONT-LOADING MEDICAL INJECTOR AND SYRINGES, SYRINGE INTERFACES, SYRINGE ADAPTERS AND SYRINGE PLUNGERS FOR USE THEREWITH, the disclosure of which is incorporated herein by reference, injection into multiple sites during certain injection procedures allows a reduction in flow rate and pressure, potentially reducing the danger of vascular damage and/or extravasation. Moreover, different fluids can be simultaneously injected into one or more injection sites on a patient wherein the fluids cannot be mixed prior to injection.

Likewise, fluids can be injected into and aspirated from a treatment region simultaneously so that the treatment region is continually exposed to the administered fluid. Such aspiration can, for example, be controlled manually. Alternatively, an injector can be used to control the aspiration. For example, a dual injector can be used wherein one of the drive members of the injector is in operative connection with an adapter of the present invention and the other drive member is used for aspiration. An example of a suitable dual injector is the SPECTRIS® injector available from Medrad, Inc. of Indianola, Pa. A dual injector is also discussed in U.S. patent application Ser. No. 10/159,592 and U.S. Pat. No. 5,494,036, the contents of which are hereby incorporated by reference.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a top view of an embodiment of injector system of the present invention in which a syringe system and an adapter are shown in cross-section and the injector is shown as transparent.

FIG. 1B illustrates a front perspective view of the adapter of FIG. 1A aligned for attachment to the attachment interface of the injector of FIG. 1A.

FIG. 2A illustrates a front view of the adapter of FIG. 1A in which the syringe system attachment is in a closed or operating position.

FIG. 2B illustrates a front view of the adapter of FIG. 1A in which the syringe system attachment is in an open or loading position.

FIG. 3 illustrates a front view of the syringe or syringe system of FIG. 1A attached to the adapter of FIG. 1A.

FIG. 4 illustrates a top view of the syringe system of FIG. 1A aligned for attachment to the adapter of FIG. 1A.

FIG. 5 illustrates a front view of multiple, single-barreled syringes attached to the adapter of FIG. 1A.

### DETAILED DESCRIPTION OF THE INVENTION

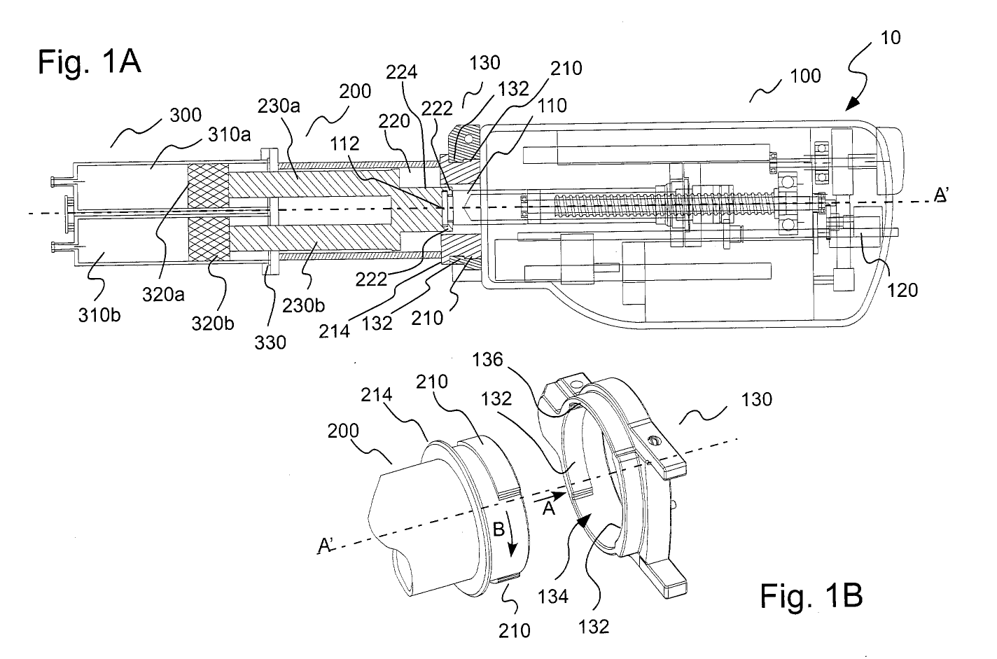

FIG. 1A illustrates an embodiment of an injector system 10 of the present invention including a powered injector 100, an adapter or intermediate connector 200 and a multiple-chamber or multiple-barrel syringe system 300 (hereinafter referred to simply as syringe 300). In the embodiment of FIGS. 1A-4, syringe 300 includes two, generally parallel, connected chambers or barrels 310a and 310b. However, as clear to one skilled in the art, the adapters of the present invention can also be used with a syringe system including a plurality of separate syringes/syringe barrels (see, for example, FIG. 5 as discussed below).

As used herein in describing injection system 10, components thereof and other embodiments of the present invention, the terms “axial” or “axially” refer generally to, for example, an axis A′ around which syringe 300 and injector drive member or piston 110 are preferably formed (although not necessarily symmetrically therearound) and to directions collinear with or parallel to axis A′. The terms “rear” or “rearward” refer generally to an axial or a longitudinal direction toward the end of injector 100 opposite the end to which syringe 300 is attached or mounted. The term “forward” refer generally to an axial or a longitudinal direction opposite of rearward. The term “radial” refers generally to a direction normal to an axis such as axis A′.

As typical with powered injectors, drive member or piston 110 of injector 100 is in operative connection with an electric motor 120 as known in the art to impart linear reciprocal motion to piston 110. Injector 100 also includes a retainer or attachment interface 130 to which a syringe or an adapter is connectible. In the embodiment of FIGS. 1A-4, injector 100 is the PROVIS® injector available from Medrad, Inc. of Indianola, Pa. Virtually any injector, as known in the art, however, is suitable for use in connection with the present invention, including those shown and described in U.S. Pat. Nos. 4,006,736, 4,677,980 and 5,383,858, the contents of which are hereby incorporated by reference.

Interface 130 can be removably or permanently mounted on the injector 100 for retaining a syringe or an adapter such as adapter 200 thereon. The syringe or adapter is, for example, releasably or removably connected to the interface 130 by means of a bayonet connection. To implement the bayonet connection, interface 130 preferably includes two retaining flanges 132 formed around an opening 134 therein (see, for example, FIG. 1B). Adapter 200 preferably includes two complementary mounting flanges 210 formed thereon for mating with the retaining flanges 132. Further, adapter 200 may include a sealing or biasing flange 214 for abutting a peripheral edge 136 of interface 130 when adapter 200 is mounted thereon.

To attach adapter 200 to injector 100, adapter 200 is inserted into interface 130 (see Arrow A in FIG. 1B) and then rotated (see Arrow B) to engage the two mounting flanges 210 with the two retaining flanges 132. A similar bayonet apparatus for front-loading a syringe or adapter onto an injector is disclosed, for example, in U.S. Pat. No. 5,383,858, the disclosure of which is incorporated herein by reference.

Adapter 200 preferably further includes an intermediate drive member 220 that is connectible to piston 110 of injector 100 to transfer motion of piston 110 to plungers 320a and 320b slidably disposed within syringe barrels 310a and 310b, respectively. Drive member 220 can, for example, include capture members 222 that cooperate with a piston flange 112 or piston 110 to removably connected piston 110 and drive member 220 in a manner similar to that described, for example, in U.S. Pat. Nos. 5,383,858, 5,873,861 and 5,947,935, the contents of which are hereby incorporated by reference.

In the embodiment of FIGS. 1A through 4, drive member 220 includes a generally central or common member 224 to which capture members 222 are attached. Common member 224 is also in operative connection with a plurality of plunger drives 230a and 230b (two in this embodiment). As illustrated in FIG. 1A, plunger drives 230a and 230b can, for example, be formed integrally with common member 224 (for example, via appropriate molding of a polymeric material as known in the art).

Plunger drives 230a and 230b form an operative connection with syringe plungers 320a and 320b, respectively, to move syringe plungers 320a and 320b within syringe barrels 310a and 310b, respectively, and inject fluid therefrom. In the embodiment of FIGS. 1A-4, plungers 320a and 320b are simply abutted by plunger drives 230a and 230b. Thus, syringe plungers 320a and 320b can be advanced by forward movement of drive member 220, but cannot be retracted. A connection (for example, a releasable connection) as known in the art can be made between plunger drives 230a and 230b and plungers 320a and 320b as known in the art (for example, via cooperating capture members and flanges as described above and in U.S. Pat. Nos. 5,383,858, 5,873,861 and 5,947,935) to allow retraction of plungers 320a and 320b upon rearward motion of drive member 220. Many other types of plunger/plunger drive connections as known in the art can also be used in connection with the present invention.

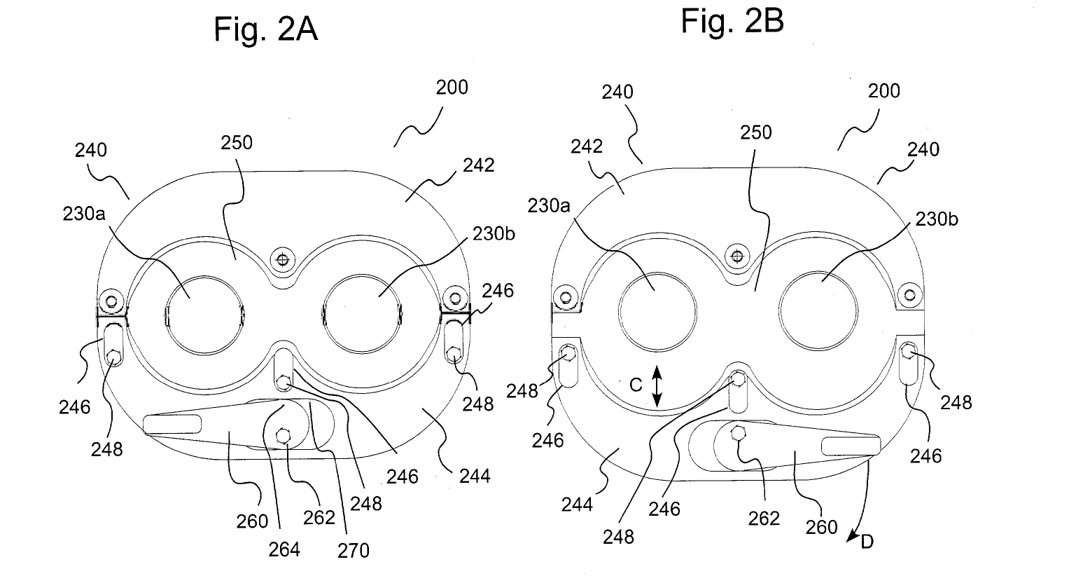

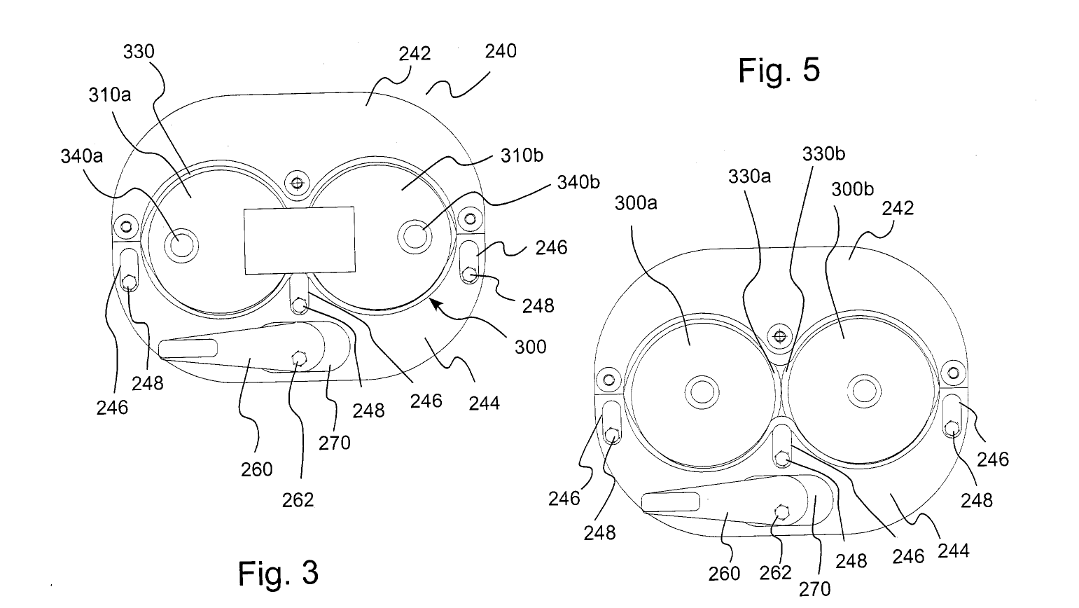

Adapter 200 and syringe 300 include cooperating attachment mechanisms to removably attach syringe 300 to adapter 200. Syringe 300 can, for example, be removably attached to adapter 300 via an attachment flange 330 extending radially outward from a rear section of syringe 300. In the embodiment of FIGS. 1A through 4, flange 330 is continuous around the periphery of a rear section of syringe 300. Alternatively, a plurality of spaced flanges or a segmented flange can be used. In the embodiment of FIGS. 1A through 4, adapter 200, includes an attachment mechanism 240 having two retaining members 242 and 244 that are attached to a forward surface 250 of adapter 200. Retaining member 242 is fixed in position relative to surface 250. Retaining member 244, however, is movable (slidable in the embodiment of FIGS. 1A through 4) in a direction toward retaining member 242 (see FIG. 2A) and away from retaining member 242 (see FIGS. 2B and 3) as represented by arrow C of FIG. 2B. In that regard, retaining member 244 includes elongated slots 246, which slide relative to post/retainers 248. The position of retaining member 244 is controlled via a rotating lever 260, which is eccentrically attached to surface 250 via a fastening member 262. When lever 260 is rotated (see arrow D of FIG. 2B) to a closed position (see FIGS. 2A and 3), a portion 264 of lever 262 abuts the periphery of a passage 270 formed in retaining member 244 to slide retaining member toward retaining member 242 (that is, toward a closed position).

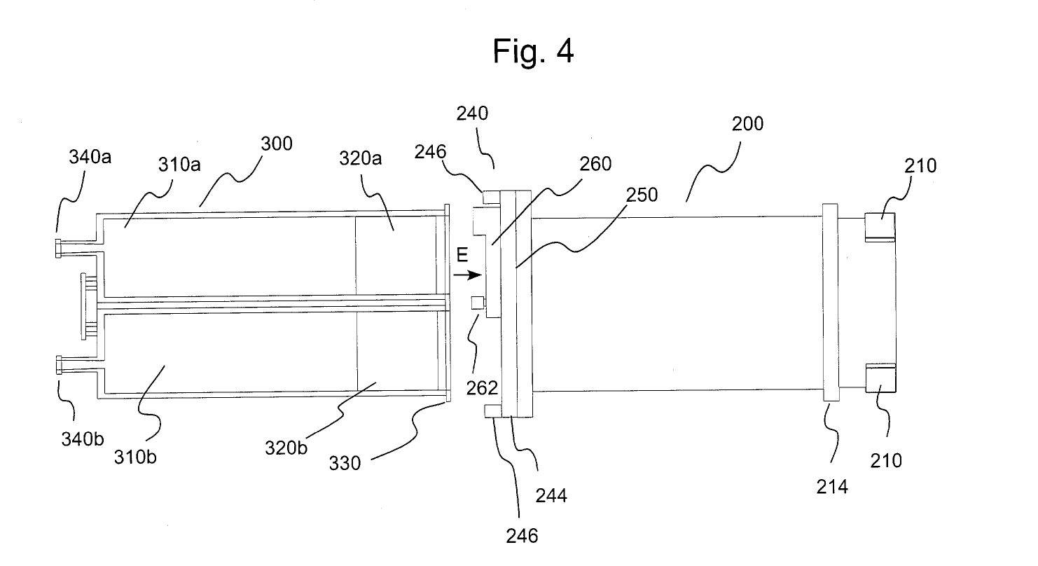

In attaching syringe 300 to adapter 200, attachment mechanism 240 is first placed in the open position as illustrated in FIG. 2B via appropriate positioning of lever 260. Syringe 300 is then aligned with adapter 300 as illustrated, for example, in FIG. 4 and inserted into attachment mechanism 240 (see arrow E of FIG. 4) so that a rear surface of flange 330 abuts surface 250. Lever 260 is then rotated to its closed position as illustrated in FIGS. 2A and 3. As illustrated, for example, in FIG. 3, radially inward portions of retaining member 242 and retaining member 244 abut flange 330 to retain syringe 300 in operative connection with adapter 200 (and therethrough in operative connection with injector 100). A radially inward portion of retaining member 242 and of retaining member 244 can, for example, be beveled to pass over and abut flange 330, when lever 260 is rotated to its closed position. Syringe 300 can be removed from connection with adapter 300 by rotating lever 260 to the open position (see FIG. 2B).

In the embodiment of FIGS. 1A through 4, each of syringe barrels 310a and 310b are of generally the same diameter and volume. In this embodiment, the same volume and/or flow rate of injection fluids will be expelled from syringe tips 340a and 340b upon forward advancement of piston 110. Injection of different volumes and/or flow rates of injection fluid from a plurality of syringe barrels can, for example, be achieved in the present invention by making one or more of such syringe barrels have different diameters and/or volumes.

FIG. 5 illustrates another embodiment of the present invention in which two syringes 300a and 300b are attached to adapter 200 via cooperation of rear flanges 330a and 330b, respectively, with retaining members 242 and 244 generally as described above.

As clear to on skilled in the art, many types of attachment mechanisms or schemes as known in the art can be used to attach the adapters of the present invention to virtually any injector. Likewise, many types of syringe or syringe system attachment mechanisms as known in the art can be used to attach one, two, three or more syringes to the adapters of the present invention.

The foregoing description and accompanying drawings set forth the preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. An adapter for connecting a syringe system having a plurality of syringe barrels to an injector having a drive member, the adapter comprising:
an injector attachment;
an intermediate drive member comprising a plurality of plunger drives; and
a syringe system attachment.

2. The adapter of claim 1 wherein each of the plurality of plunger drives is in general alignment with one of the plurality of barrels when the syringe system is attached to the adapter via the syringe system attachment.

3. The adapter of claim 1 wherein the syringe system comprises a plurality of separate syringes.

4. The adapter of claim 1 wherein the plurality of syringe barrels of the syringe system are in generally parallel attachment.

5. The adapter of claim 1 wherein the injector attachment comprises at least one flange positioned on a rear portion of the adapter.

6. The adapter of claim 1 wherein the syringe system attachment comprises at least one retaining member that is movable to move into and out of abutting engagement with the syringe system.

7. The adapter of claim 6 wherein the syringe system attachment further comprises a fixed retaining member to engage the syringe.

8. The adapter of claim 1 wherein the intermediate drive member includes a common member to which the plurality of plunger drives are in operative connection.

9. The adapter of claim 8 wherein the common member includes a drive attachment for removably attaching the intermediate drive to the drive member of the injector.

10. A system for generally simultaneous injection of a plurality of injection fluids, comprising:
a syringe system comprising a plurality of barrels, each syringe barrel having a plunger slidably disposed therein;
an injector comprising
a drive member; and
an attachment interface; and
an adapter comprising
an injector attachment cooperating with the attachment interface of the injector to attach the adapter to the injector;
an intermediate drive member comprising a plurality of plunger drives, the intermediate drive member translating at least forward motion of the drive member of the injector to the syringe plungers; and
a syringe system attachment.

11. The system of claim 10 wherein each of the plurality of plunger drives is in general alignment with one of the plurality of syringe barrels when the syringe system is attached to the adapter via the syringe system attachment

12. The system of claim 10 wherein the syringe system comprises a plurality of separate syringes.

13. The system of claim 10 wherein the plurality of syringe barrels of the syringe system are in generally parallel attachment.

14. The system of claim 10 wherein the injector attachment comprises at least one flange positioned on a rear portion of the adapter.

15. The system of claim 10 wherein the syringe system attachment comprises at least one retaining member that is movable to move into and out of abutting engagement with the syringe system.

16. The system of claim 15 wherein the syringe system attachment further comprises a fixed retaining member to engage the syringe.

17. The system of claim 10 wherein the intermediate drive member comprises a common member to which the plurality of plunger drives are in operative connection.

18. The system of claim 17 wherein the common member comprises a drive attachment for removably attaching the intermediate drive to the drive member of the injector.

19. A method of generally simultaneously injecting a plurality of fluids using an injector comprising a drive member, the method comprising:
placing an intermediate drive member in operative connection with the drive member of the injector, the intermediate drive member including a plurality of plunger drives; and
advancing the drive member of the injector to generally simultaneously advance each of the plurality of plunger drives to pressurize a fluid in each of a plurality of syringe barrels with which the plunger drives are in operative connection.

20. The method of claim 19 wherein the fluids are injected into different injection sites on a patient.

21. The method of claim 19 wherein the fluids are injected into a single injection site on a patient.

22. The method of claim 22, further comprising: aspirating fluid from the injection site during injection.
